(19) United States
(12) Patent Application Publication
(10) Pub. No.: US 2026/0335533 A1
(43) Pub. Date: Oct. 29, 2026

(54) INFUSION ASSEMBLY

(71) Applicant: Fresenius Kabi Deutschland GmbH, Bad Homburg (DE)

(72) Inventor: Steffen Baumgart, Hünfeld (DE)

(21) Appl. No.: 19/484,857

(22) PCT Filed: Jul. 3, 2024
(86) PCT No.: PCT/EP2024/068762

§ 371 Date: Nov. 14, 2025

(30) Foreign Application Priority Data
Jul. 4, 2023 (EP) 23183455.7

(51) Int. Cl. A61M 5/14; A61M 39/12; B29C 65/48; B29C 65/52; B29L 23/00

(52) U.S. Cl. CPC A61M 5/14; A61M 39/12; B29C 65/4895; B29C 65/52; B29L 2023/007

(57) ABSTRACT

An Infusion assembly comprises a tube (1), a connector (20a, 20b) comprising a connection portion (200a, 200b) configured to be engageable with the tube (1), and a joining agent (30) adapted for solvent bonding the connection portion (200a, 200b) with the tube (1), wherein the connection portion (200a, 200b) comprises a receiving surface (202a, 202b) for receiving the joining agent (30). The joining agent (30) comprises a polar solvent and a styrene block copolymer.

The invention relates to an infusion assembly, comprising a tube, a connector and a joining agent. The invention also relates to a method for joining a tube to a connector.

Infusion sets are often utilized in the medical field for administering fluids or liquids, e.g. liquid drugs, to a patient. Such infusion sets usually comprise one or more tubes for conveying the fluid to and from the patient, and one or more infusion components, such as for example a drip chamber, a pump section, filters, or adapters, each containing at least one connector to be engaged with the tube(s). Typically, such tubes are made from thermoplastic elastomer, especially polyvinylchlorides or polyurethanes, and the respective connectors are made of or comprise a thermoplastic polymer.

For the connection of the tube(s) to the connector(s), solvent bonding by means of joining agents is often used in the prior art among other technologies like for example gluing by means of cyanoacrylates since it allows to create bonds with great strength, and durability of the bond(s). Moreover, it turned out to be a relatively economic process in terms of speed and material costs. Typically, joining agents used for solvent bonding comprise solvents such as cyclohexane, cyclohexanone, methylethylketone, or mixtures out of them.

U.S. Pat. No. 10,238,854 B2 relates to a method for joining a tube to a connector with a joining agent composition including a liquid hydrocarbon.

However, there exists an ongoing demand for improving the retention force between tubing and connector to ensure a secure connection between these components for making the connection particularly durable for medical tube set assemblies.

It is an object of the instant invention to provide a reliable and durable connection between a tube and a connector in a simple manner.

This object is achieved by an infusion assembly comprising the features of claim 1.

Accordingly, an infusion assembly comprises a tube, a connector comprising a connection portion configured to be engageable with the tube, and a joining agent adapted for solvent bonding the connection portion with the tube. Herein, the connection portion comprises a receiving surface for receiving the joining agent.

According to the invention, the joining agent comprises a polar solvent and a styrene block copolymer, in particular a styrene block copolymer based thermoplastic elastomer. Utilizing a polar solvent allows for superficially dissolving, especially for a superficial softening, of the receiving surface of the connection portion. This superficial dissolving of the receiving surface can give the receiving surface itself adhesive properties and, thus, can enable particularly good adhesion properties between tube and connector.

The connection portion can be configured as a pocket or fitting. Hence, the receiving surface can be the inner surface of the pocket or the outer surface of the fitting. The joining agent may be applied to an inner diameter or to an outer diameter of the connection portion of the connector or the tube or a combination thereof. As such, by assembling the tube into the pocket or onto the fitting of the connector, the joining agent is distributed, especially spread or smeared, over the receiving surface of the connector and the corresponding surface portion of the tube and solvent bonded by means of the joining agent.

The receiving surface can be understood as the surface of the connection portion on which the joining agent is applied and/or distributed.

In an example, the joining agent comprises at most 30%, preferably 15-20%, of the styrene block copolymer. These styrene contents turned out to provide particularly strong binding forces of the joining agent.

The polar solvent may comprise tetrahydrofurane. The polar solvent optionally comprises dichloromethane, methylethylketone and/or acetone.

In an example, the connection portion comprises a taper section, in particular a chamfer, which is configured to control the amount of joining agent to be distributed at the connection portion when the tube is engaged with the connector. Due to the taper section, excess joining agent can be wiped off and/or distributed during engaging the tube and the connector with one another. This provides for a defined and evenly distributed layer of joining agent between the receiving surface of the connection portion and the corresponding surface of the tube. As such, a particularly uniform and reliable contact and adhesion between tube and connection portion is achieved. In addition, the taper section may facilitate easy assembly of the tube and the connector.

The taper section may be adapted to reduce a diameter of the connection portion. In an example, the diameter at the site of the connection portion, which gets, during assembly, in contact first with the tube, is larger than the inner diameter of the connection portion. As such, the taper section allows for striping off excess joining agent and holding it out from the receiving surface of the connection portion. Thereby, a particularly uniform and defined distribution of joining agent can be provided between tube and connection portion.

In a further example, the diameter at the site of the connection portion, which gets, during assembly, in contact first with the tube, is smaller than the outer diameter of the connection portion. As such, the taper section allows for a targeted distribution of especially excess joining agent during assembly into the tube.

Optionally, the taper section comprises one or more recesses and/or protrusions. Recesses may be, for example, elongated depressions, such as grooves. Protrusions may be, for example, elongated bulges. The recesses serve primarily the purpose of receiving excess joining agent. The protrusions may serve for wiping off and/or displacing the excess joining agent. As such, excess joining agent can be prevented from getting between the tube and the connector in an uneven manner during assembly. This provides for a particularly uniform distribution of joining agent between tube and connector and, thus, provides a particularly strong and reliable adhesion.

The recesses and/or protrusions may extend in the direction of the longitudinal extension of the connector. Particularly, the recesses and/or protrusions are elongated. The extension can be along the longitudinal extension of the connector and, optionally, along the longitudinal extension of the taper section. This allows a particularly efficient and defined striping and/or holding off of excess joining agent and, hence, allows the creation of a particularly uniform distribution of joining agent between tube and connector.

In an example, the taper section is formed at an angle of 3 to 180°, preferably at an angle of 30 to 60°. An angle in this range provides for a chamfer, which allows for a particularly efficient striping off and/or distribution of especially excess joining agent during assembly.

The connection portion may comprise a bushing for receiving an end section of the tube within an inner perimeter of the bushing, or a plug adapted to be inserted with an outer perimeter at least in part into the end section of the tube.

In this example, the receiving surface may extend at least in part along the inner perimeter of the bushing, or along the outer perimeter of the plug. The receiving surface may herein extend along a (later) contact interface between assembled tube and connection portion and, optionally, along at least a part of the taper section.

The connector may comprise at least one out of acrylonitrile butadiene styrene, methyl methacrylate acrylonitrile butadiene styrene, poly(methyl methacrylate), polycarbonate, styrene-ethylene-butadiene-styrene, styrene-butadiene-styrene, polystyrene, styrene acetonitrile and thermoplastic polyester and/or a combination of at least two thereof.

Further, the tube may comprise a thermoplastic elastomer.

The invention is also concerned with a method for joining a tube to a connector comprising the steps of providing a tube, providing a connector comprising a connection portion configured to be engageable with the tube, applying a joining agent adapted for solvent bonding the connector and the tube and comprising a polar solvent and a styrene block copolymer, in particular a styrene block copolymer based thermoplastic elastomer, to a receiving surface of the connection portion and/or at an inner surface and/or an outer surface of the tube, and engaging the tube and the connector with one another such that the tube and the connector are in fluid connection with each other.

The step of applying a joining agent may comprise applying the joining agent only to either the receiving surface, the outer surface, or the inner surface of the tube.

Alternatively, the joining agent can only be applied to (i) the receiving surface and the outer surface of the tube, or (ii) to the receiving surface and the inner surface of the tube, or (iii) to the outer surface and the inner surface of the tube.

As another alternative, the joining agent can be applied to the receiving surface, the outer surface, and the inner surface of the tube.

With regard to the advantages, reference is made to the above explanations concerning the infusion assembly.

The method may further comprise controlling the amount of joining agent distributed between the tube and the connector when the tube is engaged with the connector, by stripping of excess joining agent material at a taper section of the connection portion, in particular at a chamfer. Especially, excess joining agent may be wiped off and/or distributed.

In an example, the chamfer comprises recesses and/or protrusions for receiving and/or displacing the joining agent during engagement of the tube and the connector. As such, a particularly uniform distribution of joining agent between tube and connector and, thus, a particularly strong and reliable adhesion is enabled.

The idea underlying the invention shall subsequently be described in more detail by referring to the embodiments shown in the figures. Herein:

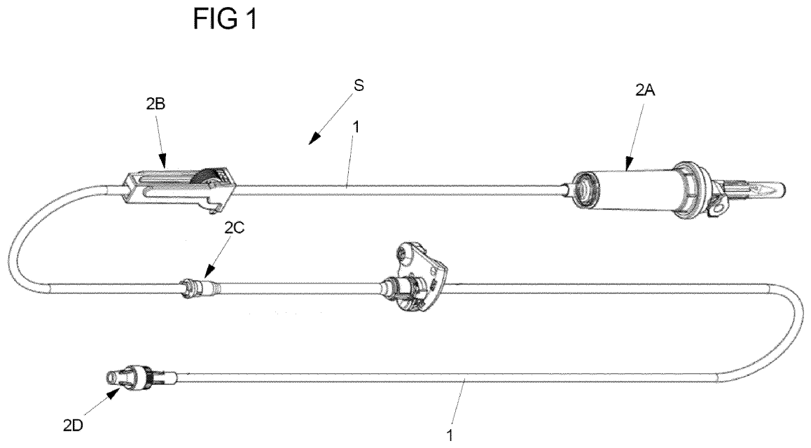

FIG. 1 shows a view of an infusion set comprising infusion components and tubes connecting the infusion components to one another;

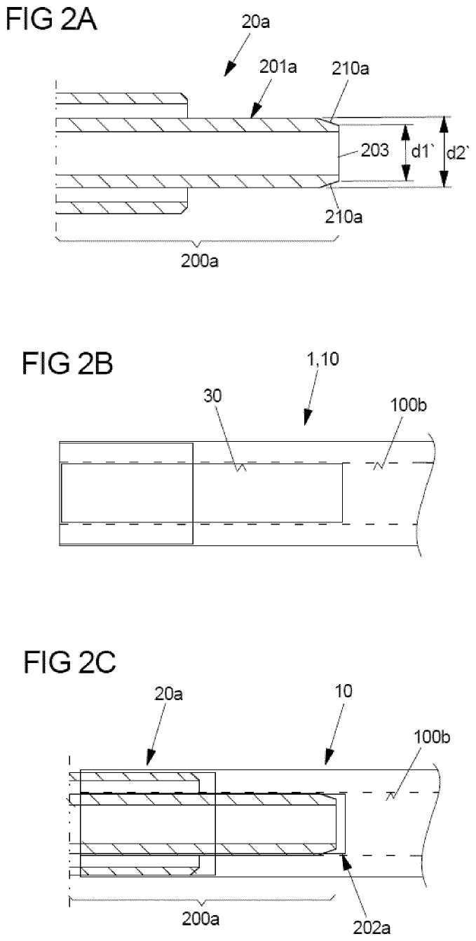

FIG. 2A shows a side view of a connection portion of connector according to a first embodiment;

FIG. 2B shows q side view of a tube section according to the first embodiment;

FIG. 2C shows a side view of the connection portion of FIG. 2A engaged with the tube of FIG. 2B according to the first embodiment;

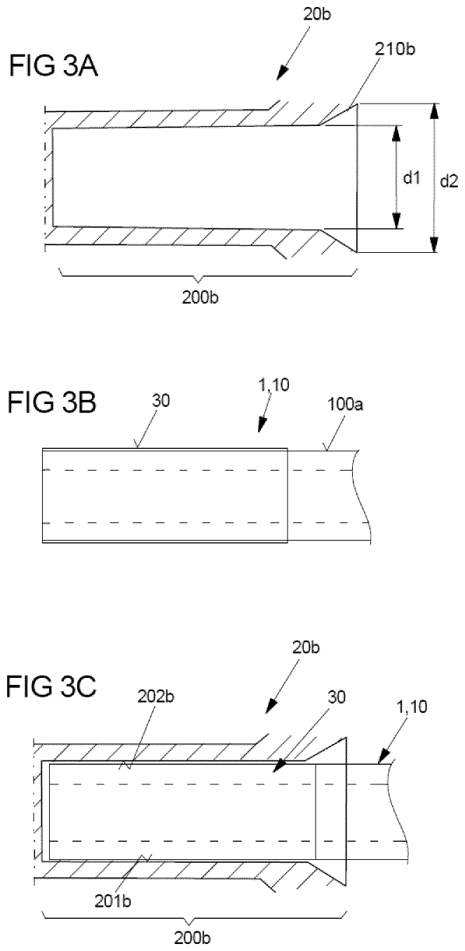

FIG. 3A shows a side view of a connection portion of the connector according to a second embodiment;

FIG. 3B shows a side view of a tube section according to the second embodiment;

FIG. 3C shows a side view of the connection portion of FIG. 3A engaged with the tube of FIG. 3B according to the second embodiment;

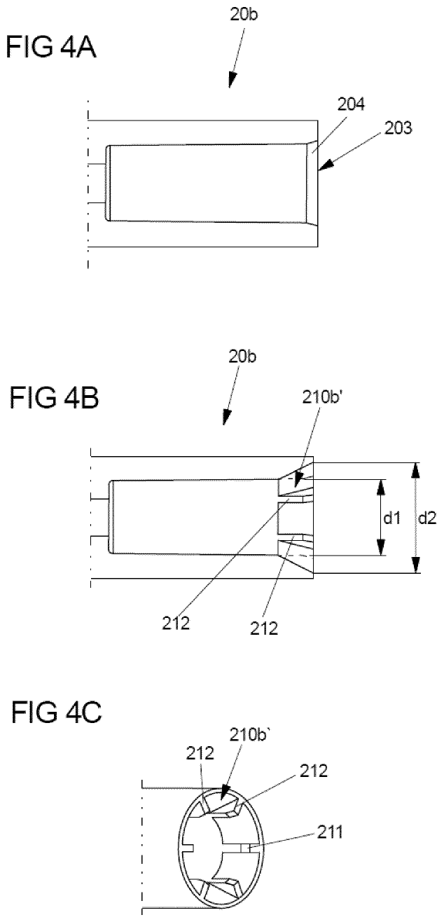

FIG. 4A shows a side view of a connection portion of a connector according to an embodiment;

FIG. 4B shows a side view of a connection portion of a connector with a taper section according to the embodiment of FIG. 4A;

FIG. 4C shows a perspective view of the taper section shown in FIG. 4B.

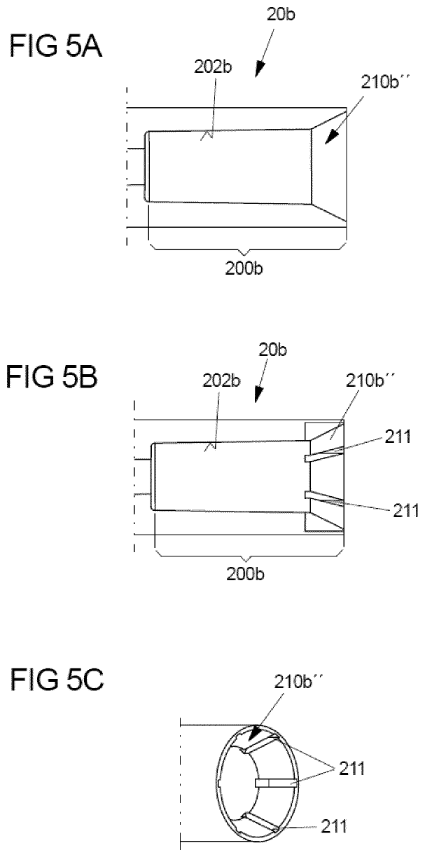

FIG. 5A shows a side view of a connection portion of a connector according to another embodiment;

FIG. 5B shows a side view of a connection portion of a connector with a taper section according to the embodiment of FIG. 5A;

FIG. 5C shows a perspective view of the taper section according to the embodiment FIG. 5B;

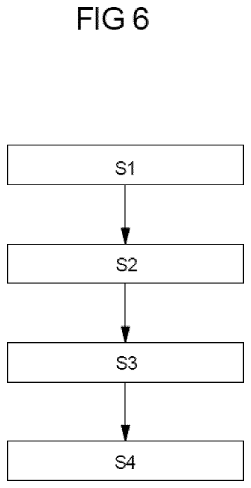

FIG. 6 shows steps of a method for joining a tube to a connector.

FIG. 1 shows an example of an infusion assembly, here a typical infusion set S is shown. The typical infusion set S includes one or more infusion components 2A-2D and at least one tube 1 connected to the one or more infusion components 2A-2D, particularly for bringing the infusion components in (fluid) connection with one another. The shown infusion set S comprises a drip chamber 2A, a pump section 2C for insertion into an infusion pump, and a Luer connector 2D as infusion components, which are all (fluidly) connected by tubes 1. The infusion set S further comprises a roller clamp 2B. The typical infusion set S may include further infusion components that are not shown in FIG. 1, e.g. Y-junctions etc., and may be formed by any combination of infusion components and tubes 1. Also, the infusion set S could comprise fewer components than the components shown in FIG. 1, such as just one tube 1 and drip chamber 2A.

FIGS. 2A and 3A show a connector 20a, 20b of an infusion component 2A-2D according to different embodiments. In the first embodiment shown in FIG. 2A, the connector 20a is illustrated as a fitting in the form of a plug, whereas FIG. 3A shows a second embodiment of the connector 20b where the connector 20b is shown as a bushing or pocket. Each connector 20a, 20b comprises a connection portion 200a, 200b to be engageable with a tube 1 (not shown in FIGS. 2A and 3A, 3B). Thereby, the connection portion 200a, 200b is the portion of the connector 20a, 20b, which is to be brought in (fluid) connection with an end section 10 of the tube 1 as shown in FIG. 2C, 3C.

In the first embodiment where the connection portion 200a of the connector 20a is shaped as a plug, the connection portion 200a comprises an (outer) perimeter 201a. Hence, for engaging the connector 20a with the tube 1, the connection portion 200a is inserted into the corresponding tube end section 10. Hereby, the surface of the outer perimeter 201a of the connection position 200a is brought into contact with the inner surface of tube end section 10 as shown in FIG. 2C.

In the second embodiment where the connection portion 200b of the connector 20b is shaped as a bushing or pocket, the connection portion 200b comprises an inner perimeter 201b. Hence, for engaging the connector 20b with the tube 1, the tube end section 10 is inserted into the corresponding connection portion 200b. Hereby, the surface of the outer surface of tube end section 10 is brought into contact with the inner perimeter 201b of the connection portion 200b as shown in FIG. 3C.

For securely and fixedly joining the tube 1 and connector 20a, 20b, a joining agent 30 is applied before engaging the tube 1 and the connector 20a, 20b with one another. The joining agent 30 can be applied to the receiving surface 202a, 202b of the connection portion 200a, 200b and/or at an inner surface 100b and/or an outer surface 100a of the tube end section 10 to be engaged with the connection portion 200a, 200b.

The joining agent 30 for solvent bonding the tube 1 and the connector 20a, 20b comprises, in the shown embodiments, a polar solvent and a styrene block copolymer, in particular in the form of a styrene block copolymer based thermoplastic elastomer.

The connection portion 200a, 200b shown in FIGS. 2C and 3C expediently comprises a receiving surface 202a, 202b. The receiving surface 202a, 202b is the surface of the connection portion 200a, 200b, on which the joining agent 30 for solvent bonding is to be applied and/or distributed. The receiving surface 202a, 202b extends along the longitudinal extension of the connection portion 200a, 200b. As shown in FIGS. 2C and 3C, the receiving surface 202a, 202b extends, at least in part, along the outer perimeter 201a of the plug, or along the inner perimeter 201b of the bushing.

The connector 20a, 20b can comprise at least one material out of acrylonitrile butadiene styrene, methyl methacrylate acrylonitrile butadiene styrene, poly(methyl methacrylate), polycarbonate, styrene-ethylene-butadiene-styrene, styrene-butadiene-styrene, polystyrene, styrene acetonitrile and thermoplastic polyester and/or a combination of at least two thereof.

FIGS. 2B and 3B show a section of the tube 1 for use in the infusion assembly according to the first and second embodiment. The tube 1 is made of or at least comprises a thermoplastic elastomer, for example a polymer, such as PVC (polyvinyl chloride), PUR (polyurethane) or PP (polypropylene). However, the tube may be also made of another material.

FIGS. 4A and 5A show a connector 20b in the form of a bushing. The connector 20b comprises at its open end site 203, where the tube end section 10 is received, a bevel 204, for example a chamfer, having a draft angle of 0-10°.

As shown in FIGS. 2A, 3A, 4B and 5B, the connector 20a, 20b further comprises a taper section 210a, 210b, 210b′, 210b″, especially a chamfer. As shown, the taper section 210a, 210b, 210b′, 210b″ comprises a first diameter d1, d1′ and a second diameter d2, d2′, the first diameter d1, d1′ being smaller than the second diameter d2, d2′. In the embodiment relating to the bushing (FIGS. 3A, 4B and 5B), the second diameter d2 corresponds to the outer diameter at the open site of the connection portion 200b and the first diameter d1 to the inner diameter of the connection portion 200b. In the embodiment relating to the plug (FIG. 2A), the first diameter d1′ corresponds to the outer diameter at the end site of the connection portion 200a, and the second diameter d2′ corresponds to a larger, along the longitudinal extension inner, diameter of the connection portion 200a.

The taper section 210a, 210b allows for striping off excess joining agent 30 and holding it out from the receiving surface 202a, 202b of the connection portion 200a, 200b. As such, excess joining agent 30 may be wiped off and/or distributed during engaging the tube 1 and the connector 20a, 20b with one another. This provides for a defined and evenly distributed layer of joining agent 30 between the receiving surface 202a, 202b of the connection portion 200a, 200b and the corresponding surface 100a, 100b of the tube 1.

The taper section 210a, 210b, 210b′, 210b″ can be formed at an angle of 3 to 180°, preferably at an angle of 30 to 60°.

FIGS. 4B and 5B show a side view of a connector 20b. The connector 20b comprises a plurality of recesses 211 and/or protrusions 212. Recesses 211 are shown as elongated depressions, such as grooves. Protrusions 212 are shown as elongated bulges. The recesses 211 serve primarily for receiving excess joining agent 30. The protrusions 212 serve primarily for wiping off and/or displacing the excess joining agent 30. As such, excess joining agent 30 can be prevented from getting between the tube 1 and the connector 20a, 20b in an uneven manner during assembly. This enables a particularly uniform distribution of joining agent 30 between tube 1 and connector 20a, 20b and, thus, for a particularly strong and reliable adhesion.

FIGS. 4C and 5C show perspective views of taper sections 210b′, 210b″. The taper section 210b′ shown in FIG. 4C comprises recesses 211 and protrusions 212. The taper section 210b″ shown in FIG. 5C comprises recesses 211 only.

The recesses 211 and/or protrusions 212 are elongated and extend in the direction of the longitudinal extension of the connection portion 200a, 200b and, hence, along the longitudinal extension of the connector 20a, 20b.

By means of the taper section 210a, 210b, 210b′, 210b″, and especially by means of the recesses 211 and/or protrusions 212, it is possible to control the amount of joining agent 30 to be distributed at the connection portion 200a, 200b when the tube 1 is engaged with the connector 20a, 20b. As such, a particularly uniform and reliable contact and adhesion between tube 1 and connection portion 200a, 200b is achieved.

In addition, the taper section 210a, 210b, 210b′, 210b″ can facilitate assembly of the tube 1 and the connector 20a, 20b.

FIG. 6 shows a method for joining a tube 1 to a connector 20a, 20b. The method comprises the following steps:

In a first step S1, a tube 1 is provided. In a second step S2, a connector 20a, 20b is provided comprising a connection portion 200a, 200b configured to be engageable with the tube 1. In a third step S3, a joining agent 30 adapted for solvent bonding the connector 20a, 20b and the tube 1 and comprising a polar solvent and a styrene block copolymer, in particular a styrene block copolymer based thermoplastic elastomer, is applied to a receiving surface 202a, 202b of the connection portion 200a, 200b and/or at an inner surface 100b and/or at an outer surface 100a of the tube 1. In a fourth step S4, the tube 1 and the connector 20a, 20b are engaged with one another such that the tube 1 and the connector 20a, 20b are in fluid connection with each other.

In step S3, the joining agent 30 may be applied solely to the receiving surface 202a, 202b of the connection portion 200a, 200b. Alternatively or in addition, the joining agent 30 may be applied at the inner surface 100b of the tube 1, at the outer surface 100a of the tube 1, or at both of them.

### LIST OF REFERENCE NUMERALS

1 tube
10 tube end section
100a outer surface
100b inner surface
2A-2D infusion component
20a, 20b connector
200a, 200b connection portion
201a outer perimeter
201b inner perimeter
202a, 202b receiving surface
210a, 210b, 210b′, 210b″ taper section
211 recesses
212 protrusions
30 joining agent
d1, d2, d1′, d2′ diameter
S infusion set
S1-S4 steps

1. An infusion assembly, comprising:
a tube,
a connector comprising a connection portion configured to be engageable with the tube, and
a joining agent adapted for solvent bonding the connection portion with the tube,
wherein the connection portion comprises a receiving surface for receiving the joining agent, and
wherein the joining agent comprises a polar solvent and a styrene block copolymer.

2. The infusion assembly according to claim 1, wherein the joining agent comprises at most 30% of the styrene block copolymer.

3. The infusion assembly according to claim 1, wherein the polar solvent comprises tetrahydrofurane, dichloromethane, methylethylketone, or acetone, or a combination of at least two thereof.

4. Infusion The infusion assembly according to claim 1, wherein the connection portion comprises a taper section configured to control the amount of joining agent to be distributed at the connection portion when the tube is engaged with the connector.

5. The infusion assembly according to claim 4, wherein the taper section is adapted to reduce a diameter of the connection portion.

6. The infusion assembly according to claim 4, wherein the taper section comprises one or more recesses and/or protrusions.

7. The infusion assembly according to claim 6, wherein the recesses and/or protrusions extend in a direction of a longitudinal extension of the connector.

8. The infusion assembly according to claim 4, wherein the taper section is formed at an angle of 3 to 180°.

9. The infusion assembly according to claim 1, wherein the connection portion comprises a bushing for receiving an end section of the tube within an inner perimeter of the bushing, or a plug adapted to be inserted with an outer perimeter at least in part into the end section of the tube.

10. The infusion assembly according to claim 9, wherein the receiving surface extends at least in part along the inner perimeter of the bushing, or along the outer perimeter of the plug.

11. The infusion assembly according to claim 1, wherein the connector comprises at least one out of acrylonitrile butadiene styrene, methyl methacrylate acrylonitrile butadiene styrene, poly(methyl methacrylate), polycarbonate, styrene-ethylene-butadiene-styrene, styrene-butadiene-styrene, polystyrene, styrene acetonitrile and thermoplastic polyester and a combination of at least two thereof.

12. The infusion assembly according to claim 1, wherein the tube comprises a thermoplastic elastomer.

13. A method for joining a tube to a connector comprising the steps of:
providing the tube,
providing the connector comprising a connection portion configured to be engageable with the tube,
applying a joining agent adapted for solvent bonding the connector and the tube and comprising a polar solvent and a styrene block copolymer to a receiving surface of the connection portion and/or at an inner surface and/or an outer surface of the tube,
engaging the tube and the connector with one another such that the tube and the connector are in fluid connection with each other.

14. The method according to claim 13, further comprising controlling the amount of joining agent distributed between the tube and the connector when the tube is engaged with the connector by stripping off excess joining agent at a taper section of the connection portion.

15. The method according to claim 14, wherein the taper section comprises recesses and/or protrusions for receiving and/or displacing the joining agent during engagement of the tube and the connector.

16. The infusion assembly according to claim 2, wherein the joining agent comprises 15 to 20% of the styrene block copolymer.

17. The infusion assembly according to claim 4, wherein the taper section comprises a chamfer.

18. The infusion assembly according to claim 8, wherein the taper section is formed at an angle of 30 to 60°.
